(19) United States
(12) Patent Application Publication
(10) Pub. No.: US 2006/0099257 A1
(43) Pub. Date: May 11, 2006

(54) Controlled drug delivery systems providing variable release rates

(76) Inventors: John Richard Langridge, Sint Oedenrode (NL); Janine Clare Collins, Sheerness (GB); Wel Tian, Manrly (GB)

Correspondence Address: MCANDREWS HELD & MALLOY, LTD, 500 WEST MADISON STREET, SUITE 3400, CHICAGO, IL, 60661, US

(21) Appl. No.: 10/484,734

(22) PCT Filed: Jul. 18, 2002
(86) PCT No.: PCT/GB02/03286

§ 371 Date: Jan. 17, 2006

(30) Foreign Application Priority Data
Jul. 19, 2001 (GB) 0117618.9

(51) Int. Cl. A61K 9/24

(52) U.S. Cl. 424/472

(57) ABSTRACT

A controlled release dosage form with variable release rates comprising: 1) a bilayer or multilayer tablet core in which at least one of the layers contains one or more pharmaceutically active ingredients and at least one of the layers contains one or more rate controlling polymers; 2) a substantially insoluble casing extended over the tablet core covering the majority of tablet surface but leaving a portion of one layer of the table core exposed (exposed layer), the casing resulting from electrostatic deposition of a powder comprising fusible particles onto the tablet core and fusing the particles to form a thin film.

The present invention relates to a drug delivery system that releases one or more active materials at controlled and variable rates into a biological fluid, in particular, the fluid of the gastrointestinal tract.

Tablets are often the preferred means of administering medicine to a patient. A conventional immediate release tablet releases the drug active in the body, rapidly reaching a maximum concentration then decaying expeditiously until the next administration. This method often leads to t4e peaks and troughs of drug concentration in the blood and requires frequent administration of tablets. Consequently, this could lead to either exacerbated harmful side effects at high concentrations or diminished therapeutic effects at low concentrations. These effects can become acute with actives of relatively short biological half life. Another disadvantage of immediate release dosage form is that a frequent dosing regime is required, thereby causing problems of patient compliance. To counter these, controlled release dosage forms that release actives at a constant rate over a defined period of time (zero order release) have been frequently employed. A range of matrix forming natural and synthetic polymers is employed to prolong drug release, for example, xanthan gum, galactomannan polymers, alginate, cellulose derivatives (methycellulose, hydroxypropylcellulose and hydroxy propyl methyl cellulose etc.), acrylic and methacrylic co-polymers and combinations thereof. The diverse range of polymers enables formulators to obtain the desired release profile of drug actives despite the vast differences in the physicochemical properties of these actives.

More recently, the roles of circadian rhythms in certain physiological functions have gained increased recognition. It is known that many symptoms and onset of disease occur during specific time periods of the day, for example, gout, gall bladder and peptic ulcer attacks are most frequent at night; angina pectoris, sudden cardiac death, ventricular arrhythmia, stroke all occur most frequently in the morning (Smolensky, M. H. (2001), CNS Spectrum, Volume 16, Pages 467-482). This knowledge has led to the development of chronotherapeutics that requires a more “programmable” release of drug in the human body to enhance the therapeutic effect and to minimise the adverse effects of the drug.

GB2241485 claims a pulsed release device for releasing the contents of a capsule into an aqueous medium that comprises a water impermeable capsule having at least one orifice which is characterised in that the orifice is closed with a water soluble or water dispersible plug.

U.S. Pat. No. 6,303,144 discloses a controlled release preparation containing at least one kind of a pharmaceutically active ingredient, a male piece and a female piece, the pieces fitting together to enclose the active substance therein, wherein the male piece is made from a material that gels in the intestinal juice.

U.S. Pat. No. 464,633 claims a pharmaceutical tablet for oral administration suitable to release the active substance after a definite period of time, consisting essentially of: a core containing the active substance and a polymeric substance which swells and/or gels and/or erodes on contact with water; a layer applied externally to said core by a compression process with a thickness of 0.2-4.5 mm which allows the release of said active after 2-3 hours.

U.S. Pat. No. 6,183,778 claims an oral dosage form in the form of a tablet, capable of providing one or more pharmaceutically active substances in two or more different releases, the dosage form comprising at least three layers of specific geometric shape, wherein the dosage form comprises: a) a first layer, from which there occurs a first release of at least one pharmaceutically active substance, wherein the release is characterised as an immediate release or a controlled release, the layer comprising substances which swell or solubilise when contacted with aqueous liquids; b) a second layer from which there occurs a second release of at least one pharmaceutically active substances, wherein at least one pharmaceutically active substance is the same as or different from the at least one pharmaceutically active substance released from the first layer in the first release, wherein the second release is characterised as controlled release, the second layer comprising substances that swell, or erode, or are gellable when contacted with aqueous liquid; and c) a third layer at least partially coating one or more free surfaces of the second layer, the third layer comprising substances that swell, or erode, or are gellable when contacted with aqueous liquid.

U.S. Pat. No. 5,681,583 discloses a multilayered controlled-release solid pharmaceutical composition in tablet form suitable for oral administration comprising at least two layers containing active material in association with excipients and additives. One layer of the tablet releases a portion of the drug quickly while the other layer and optionally further layers release portions of the drug more gradually.

U.S. Pat. No. 5,213,808 discloses an article for controlled delivery of an active substance into an aqueous phase has a first layer containing an active substance, and a second layer of a crystalline polymer matrix and a non-ionic surface active agent, the second layer also containing the same or different active substance substantially homogeneously dispersed therein. The article enables release of a drug active at a constant plateau level followed by a pulse of drug after a predetermined time.

U.S. Pat. No. 5,004,614 discloses controlled release devices having a core including an active agent and an outer coating which is substantially impermeable to the entrance of an environmental fluid and substantially impermeable to the release of the active agent during a dispensing period allow the controlled release of the active agent through an orifice in the outer coating. The coating thickness, the position, number and the sizes of the orifices are the key variables influencing the release profile.

WO 921445 discloses that electrostatic deposition may be used to apply a coating of controlled thickness and may be employed for a medicinal product containing a drug that is to be instantaneously released when administered or that is to be the subject of controlled or modulated release, such control of modulation being achieved from the nature of the coating and/or from the nature of core. Where the desired form of release is to be achieved by characteristics of the coating, it may be preferred to leave one portion of the product uncoated or coated with different material. In the case of a tablet having faces at opposite ends connected by a cylinder side wall, the portion that is uncoated or coated with different material may be one of the faces of the tablet, a small portion of one of the faces or a side wall of the tablets. However, there is no disclosure as to whether or how variable release rates profile can be achieved.

In accordance with the present invention there is provided controlled release dosage form with variable release rates comprising:
1) a bilayer or multilayer tablet core in which at least one of the layers contains one or more pharmaceutically active ingredients and one or more of the layers contains one or more rate controlling polymers
2) a substantially insoluble casing extended over the tablet core covering the majority of tablet surface but leaving a portion of one layer of the tablet core exposed, the casing resulting-from electrostatic deposition of a powder comprising fusible particles onto the tablet core and fusing the particles to form a thin film.

The invention provides a simple and effective means of producing a pharmaceutical dosage form having variable release rate profiles for one or more pharmaceutical active agents.

It has been surprisingly found that a pharmaceutical dosage form having controlled release of an active ingredient at variable rates can be obtained by electrostatic application of a thin film on the selected surface of a bilayer or multilayer tablet. Furthermore, there are no needs for a specially designed geometric shape, the mechanical removal of a portion of film coating at a defined position with a defined surface area, or the presence of specific matrix forming polymers.

The release profile of an active ingredient from the electrostatically coated tablets does not require the application of a thick film nor rely on the controlled thickness so long as a complete and uniform coating within the defined area is obtained.

The release profile of a pharmaceutical active can be determined by standard US Pharmacopoeia method using either a basket stirring element (apparatus I) or a paddle stirring element (apparatus II). Vankel™ 7000 dissolution apparatus (Apparatus II) was used for the present invention. The assembly consists of the following: a covered vessel made of glass or other inert, transparent material; a motor; a paddle formed from a blade and a shaft. The shaft is positioned so that its axis is not more than 2 mm at any point from the vertical axis of the vessel and rotates smoothly without significant wobble. The vertical centre line of the blade passes through the axis of the shaft so that the bottom of the blade is flush with the bottom of the shaft. The distance of 25±2 mm between the paddle and the inside bottom of the vessel is maintained during the test.

The vessel is partially immersed in a suitable waterbath which maintains the temperature inside the vessel at 37±0.5° C. during the test and keeping the bath fluid in constant, smooth motion. The vessel is cylindrical, with a hemispherical bottom. Its sides are flanged at the top. A fitted cover may be used to retard evaporation. Demineralised water is added to the vessel. The dosage unit (one single tablet) is allowed to sink to the bottom of the vessel before the rotation of the blade is started. The stirring rate is set at 50 rpm. The released active ingredient with time is measured by a suitable means e.g. u.v. analysis, HPLC etc. and expressed as percentage release (w/w) of the total weight of active ingredient.

In one embodiment according to the present invention the pharmaceutical dosage form has increased release rates over a definite period of time, where the exposed layer contains a lower amount of active material and/or has a slower release rate than the enclosed layer. The pharmaceutical dosage form may release its active ingredient over a prolonged period of time. Preferably a substantially complete release (i.e. 70%) of the pharmaceutical active ingredient is achieved after at least 4 hours. More preferably, a substantially complete release (i.e. 70%) of the pharmaceutical active is achieved after 6 hours.

The pharmaceutical dosage form releases the active ingredient over a first period at a slower rate than a subsequent second period. Preferably, the release rate during the second period is at least 50% greater than the first period; more preferably, the release rate during the second period is at least 75% greater than the first period. Preferably, the first period extends to at least 1 hour; more preferably the first period extends to at least 2 hours.

In a further embodiment of the invention the pharmaceutical dosage form has a delayed release profile over a definite period of time, where the exposed layer contains no active material, but contains one or more rate controlling polymers. Preferably, substantially no active ingredient, e.g. less than 10% of the active ingredient is released after at least 1 hour; more preferably less than 10% of the active ingredient is released after at least 2 hours.

In a further embodiment of the invention the pharmaceutical dosage form initially releases a first pharmaceutically active agent at a rapid release rate (fast phase), followed by the release of the same or second pharmaceutically active agent or at a slower rate, where the exposed layer contains one or more active ingredients, which can be the same or different from the active ingredient (s) present in the enclosed layer and one or more rate controlling polymers are present in the enclosed layer, but are absent in the exposed layer. Preferably the release of the first ingredient or the fast release phase is substantially completed within 40% of the entire dissolution period; more preferably, the release of the first ingredient or the fast release phase is substantially completed within 30% of the entire dissolution period.

The casing extending over the tablet core results from the electrostatic deposition of a powder comprising fusible particles. This technique allows the formation of a thin, continuous casing over the tablet core. Although the release profile does not depend on the coating thickness, it is of importance that a continuous and complete coverage is applied in order to minimise pore formation. Typically this requires the deposition of several layers of powdered material (the powders have a mean diameter of 10 μm) to give a coating thickness of at least 20 μm after fusion. Generally the average thickness of the casing is in the range 20 to 50 μm. In general, the casing will cover from 0 to 99% of the surface area of the exposed layer. Generally the coating results in a weight gain of less than 5%, often less than 4% and frequently less than 3% by weight of the tablet core.

The shape of the tablet core is not critical since the electrostatic deposition of powder can readily be achieved over a variety of shaped bodies. The tablet core is conveniently formed by conventional tableting techniques e.g. compression of powder and/or granules, although other moulding techniques may be employed. A convenient tablet core has a circular cross-section and two major opposing surfaces which may be, for example, planar, planar with a bevelled edge, concave, convex etc. The insoluble casing may conveniently extend over one of the major surfaces and the side wall leaving the other major surface exposed.

The tablet core comprises at least one adjuvant and a pharmaceutically active ingredient. Generally the adjuvant will comprise a binder. Suitable binders are well known and include acacia, alginic acid, carboxymethylcellulose, hydroxyethylcellulose, hydroxypropylcellulose, dextrin, ethylcellulose, gelatin, glucose, guar gum, hydrogenated vegetable oil, hydroxypropylmethylcellulose, magnesium aluminium silicate, maltodextrin, methylcellulose, polyethylene oxide, povidone, sodium alginate and hydrogenated vegetable oils.

The tablet core preferably comprises a release rate controlling additive. For example, the drug may be held within a hydrophobic polymer matrix so that it is gradually leached out of the matrix upon contact with body fluids. Alternatively, the drug may be held within a hydrophilic matrix which gradually dissolves in the presence of body fluid.

Suitable release rate controlling polymers include polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, zein etc.

The tablet core may comprise other conventional tableting ingredients, including diluents, disintegrants, lubricants, wetting agents, glidants, surfactants, release aids, colourants, gas producers, etc. Suitable diluents include lactose, cellulose, dicalcium phosphate, sucrose, dextrose, fructose, xylitol, mannitol, sorbitol, calcium sulphate, starches, calcium carbonate, sodium carbonate, dextrates, dextrin, kaolin, lactitol, magnesium carbonate, magnesium oxide, maltitol, maltodextrin and maltose. Suitable lubricants include magnesium stearate and sodium stearyl fumarate. Suitable glidants include colloidal silica and talc.

Suitable wetting agents include sodium lauryl sulphate and docusate sodium. Suitable gas producers include sodium bicarbonate and citric acid.

The pharmaceutically active ingredient may be selected from a wide range of substances which may be administered orally. Suitable ingredients include acid-peptic and motility influencing agents, laxatives antidiarrhoeials, colorectal agents, pancreatic enzymes and bile acids, antiarrhythmics, antianginals, diuretics, anti-hypertensives, anti-coagulants, anti-thrombotics, fibrinolytics, haemostatics, hypolipidaemic agents, anti-anaemia and neurotropenia agents, hypnotics, anxiolytics, anti-psychotics, anti-depressants, anti-emetics, anti-convulsants, CNS stimulants, analgesics, anti-pyretics, anti-migraine agents, non-steroidal anti-inflammatory agents, anti-gout agents, muscle relaxants, neuro-muscular agents, steroids, hypoglycaemic agents, hyperglycaemic agents, diagnostic agents, antibiotics, anti-fungals, anti-malarials, anti-virals, immunosuppressants, nutritional agents, vitamins, electrolytes, anorectic agents, appetite suppressants, bronchodilators, expectorants, anti-tussives, mucolytic, decongestants, anti-glaucoma agents, oral contraceptive agents, diagnostic and neoplastic agents.

The electrostatic application of powder material to a substrate is known. Methods have already been developed in the fields of electrophotography and electrography and examples of suitable methods are described, for example, in Electrophotography and Development Physics, Revised Second Edition, by L. B. Schein, published by Laplacian Press, Morgan Hill Calif. The electrostatic application of powder material to a solid dosage form is known and techniques are disclosed, for example, in GB9929946.3, WO92/14451, WO96/35413, WO96/35516 and PCT/GB01100425, and British Patent Application No. 9929946.3.

For example, WO92/14451 describes a process in which the cores of pharmaceutical tablets are conveyed on an earthed conveyor belt and electrostatically charged powder is deposited on the cores to form a powder coating on the surface of the cores.

A powder material for electrostatic application to a substrate should have certain properties. For example, the electrical properties of the powder material should be such as to make the powder material suitable for electrostatic application, and other properties of the powder material should be such that the material can be secured to the substrate once electrostatic application has taken place.

WO96/35413 describes a powder material which is especially suitable for electrostatic application to a poorly-conducting (non-metal) substrate such as a pharmaceutical tablet. Because it may be difficult to find a single component capable of providing the powder material with all the desired properties, the powder material comprises a number of different components which together are capable of providing the material with all or at least as many as possible of the desired properties, the components being co-processed to form “composite particles”. For example, the powder material may comprise composite particles including one component which is fusible to form a continuous film on the surface of the substrate, and another component which has desirable electrical properties.

A potential disadvantage of the above mentioned powder materials, however, is that they are not readily adaptable to changes in formulation. The formulation of a powder material may be changed for a number of different reasons. For example, if the material is a coloured material, there may be a change in the colourant, or if the material is an active material, for example a physiologically active material there may be a change in the type of active material, or in the concentration of that active material. Because all the components of the powder material are intimately mixed, any change in the components will alter the material's electrical properties and hence its performance in electrostatic application. Whenever there is a change in formulation, it may therefore be necessary, for optimum performance, to adjust the content of the component(s) that make the material suitable for electrostatic application, or perhaps even to use a different component.

PCT/GB01/00425 discloses a method of electrostatically applying a powder material to a substrate, wherein at least some of the particles of the material comprise a core and a shell surrounding the core, the core and the shell having different physical and/or chemical properties.

Where the particles of the powder material comprise a core and a shell surrounding the core, it is possible to place those components which are likely to be altered, for example colourant in the core, and to provide a more universal shell composition which is suitable for use with various core compositions, so that alterations may be made to the components that are in the core without substantially affecting the overall suitability of the powder material; thus, the shell ensures that the change in composition of the core does not affect the performance of the material in electrostatic application. Accordingly, alterations to one component of the powder material may be made with minimum alteration in the amounts of other components.

Generally, the powder material includes a component which is fusible, and that component may be present in the shell or in the core or in both the shell and the core. Advantageously, the fusible component is treatable to form a continuous film coating. Examples of suitable components are as follows: polyacrylates, for example polymethacrylates; polyesters; polyurethanes; polyamides, for example nylons; polyureas; polysulphones; polyethers; polystyrene; polyvinylpyrrolidone; biodegradable polymers, for example polycaprolactones, polyanhydrides, polylactides, polyglycolides, polyhydroxybutyrates and polyhydroxyvalerates; sugars, for example lactitol, sorbitol xylitol, galactitol, maltitol, sucrose, dextrose, fructose, xylose and galactose; hydrophobic waxes and oils, for example vegetable oils and hydrogenated vegetable oils (saturated and unsaturated fatty acids) e.g. hydrogenated castor oil, carnauba wax, and beeswax; hydrophilic waxes; polyalkenes and polyalkene oxides; polyethylene glycol. Clearly there may be other suitable materials, and the above are given merely as examples. One or more fusible materials may be present. Preferred fusible materials generally function as a binder for other components in the powder.

In general the powder material should contain at least 30%, usually at least 35%, advantageously at least 80%, by weight of material that is fusible, and, for example, fusible material may constitute up to 95%, e.g. up to 85%, by weight of the powder. Wax, if present, is usually present in an amount of no more than 6%, especially no more than 3% by weight, and especially in an amount of at least 1% by weight, for example 1 to 6%, especially to 1 to 3%, by weight of the powder material.

Of the materials mentioned above, polymer binders (also referred to as resins) should especially be mentioned. Examples include polyvinylpyrrolidone, hydroxypropyl cellulose, hydroxypropyl methylcellulose phthalate, hydroxypropyl methylcellulose acetate succinate and methacrylate polymers, for example an ammonio-methacrylate copolymer, for example those sold under the name Eudragit.

Often resin will be present with a wax as an optional further fusible component in the core; the presence of a wax may, for example, be useful where fusing is to take place by a contact system for example using a heated roller, or where it is desired to provide a glossy appearance in the fused film. The fusible component may comprise a polymer which is cured during the treatment, for example by irradiation with energy in the gamma, ultra violet or radio frequency bands. For example, the core may comprise thermosetting material which is liquid at room temperature and which is hardened after application to the substrate.

Preferably, the powder material includes a material having a charge-control function. That functionality may be incorporated into a polymer structure, as in the case of Eudragit resin mentioned above, and/or, for a faster rate of charging, may be provided by a separate charge-control additive. Material having a charge-control function may be present in the shell or in the core or in both shell and core. Examples of suitable charge-control agents are as follows: metal salicylates, for example zinc salicylate, magnesium salicylate and calcium salicylate; quaternary ammonium salts; benzalkonium chloride; benzethonium chloride; trimethyl tetradecyl ammonium bromide (cetrimide); and cyclodextrins and their adducts. One or more charge-control agents may be used. Charge-control agent may be present, for example, in an amount of up to 10% by weight, especially at least 1% by weight, for example from 1 to 2% by weight, based on the total weight of the powder material.

The powder material may also include a flow aid. The flow aid reduces the cohesive and/or other forces between the particles of the material to improve the flowability of the powder. Suitable flow aids (which are also known as “surface additives”) are, for example, as follows: colloidal silica; metal oxides, e.g. fumed titanium dioxide, zinc oxide or alumina; metal stearates, e.g. zinc, magnesium or calcium stearate; talc; functional and non-functional waxes, and polymer beads, e.g. poly-methyl methacrylate beads, fluoropolymer beads and the like. Such materials may also enhance tribocharging. A mixture of flow aids, for example silica and titanium dioxide, should especially be mentioned. The powder material may contain, for example, 0 to 3% by weight, advantageously at least 0.1%, e.g. 0.2 to 2.5%, of surface additive flow aid.

Often the powder material includes a colourant and/or an opacifier. When the powder comprises a core and shell such components are preferably present in the core. Examples of suitable colourants and opacifiers are as follows: metal oxides, e.g. titanium dioxide, iron oxides; aluminium lakes, for example, indigo carmine, sunset yellow and tartrazine; approved food dyes; natural pigments. A mixture of such materials may be used if desired. Opacifier preferably constitutes no more than 50%, especially no more than 40%, more especially no more than 30%, for example no more than 10% by weight of the powder material, and may be used, for example, in an amount of at least 5% by weight of the powder. Titanium dioxide is an especially useful opacifier, providing white colour and having good hiding power and tinctorial strength. Colourant present with opacifier may, for example, constitute no more than 10%, preferably from 1 to 5%, by weight of the powder. If there is no opacifier, the colourant may be, for example, 1 to 15%, e.g. 2 to 15%, especially 2 to 10%, by weight of the powder. To achieve optimum colour, amounts of up to 40% by weight of colourant may be needed in some cases, for example if inorganic pigments, e.g. iron oxides, are used. However, the powder material usually contains, for example, from 0 to 25% by weight in total of colourant and/or opacifier.

The powder material may also include a dispersing agent, for example a lecithin. The dispersing agent is preferably present with the colourant/opacifier (that is, preferably in the core), serving to improve the dispersion of the colourant and opacifier, more especially when titanium dioxide is used. The dispersing component is preferably a surfactant which may be anionic, cationic or non-ionic, but may be another compound which would not usually be referred to as a “surfactant” but has a similar effect. The dispersing component may be a co-solvent. The dispersing component may be one or more of, for example, sodium lauryl sulphate, docusate sodium, Twines (sorbitan fatty acid esters), polyoxamers and cetostearyl alcohol. Preferably, the powder material includes at least 0.5%, e.g. at least 1%, for example from 2% to 5%, by weight of dispersing component, based on the weight of the powder material. Most often it is about 10% by weight of the colourant and opacifier content.

The powder material may also include a plasticiser, if necessary, to provide appropriate rheological properties. A plasticiser may be present in the core and/or the shell, but usually, if present, a plasticiser is included with resin used for the core to provide appropriate rheological properties, for example for preparation of the core by extrusion in a melt extruder. Examples of suitable plasticisers include polyethylene glycols, triethyl citrate, acetyltributyl citrate, acetyltriethyl citrate, tributyl citrate, diethyl phthalate, dibutyl phthalate, dimethyl phthalate, dibutyl sebacate and glyceryl monostearate.

A plasticiser may be used with a resin in an amount, for example, of up to 50% by weight of the total of that resin and plasticiser, the amount depending inter alia on the particular plasticisers used. The powder may contain an amount of up to 50% by weight of plasticiser.

The powder coating material may further include one or more taste modifiers, for example aspartame, acesulfame K, cyclamates, saccharin, sugars and sugar alcohols or flavourings. Preferably there is no more than 5%, more preferably no more than 1%, of flavouring based on the weight of the powder material, but larger or smaller amounts may be appropriate, depending on the particular taste modifier used.

If desired the powder material may further include a filler or diluent. Suitable fillers and diluents are essentially inert and low cost materials with generally little effect on the colour or other properties of the powder. Examples are as follows: alginic acid; bentonite; calcium carbonate; kaolin; talc; magnesium aluminium silicate; and magnesium carbonate.

The particle size of the powder material has an important effect on the behaviour of the material in electrostatic application. Although materials having a small particle size are recognised as having disadvantages such as being more difficult to produce and to handle by virtue of the material's cohesiveness, such material has special benefits for electrostatic application and the benefits may more than counter the disadvantages. For example, the high surface to mass ratio provided by a small particle increase the electrostatic forces on the particle in comparison to the inertial forces. Increasing the force on a particle has the benefit of increasing the force that causes it to move into contact with the substrate, whilst a reduction in the inertia reduces the force needed to accelerate a particle and reduces the likelihood of a particle arriving at the substrate bouncing back off the substrate. However, very small particle sizes may not be achievable where the coating material comprises a high proportion of a particular ingredient, for example a high proportion of active material.

Preferably, at least 50% by volume of the particles of the material have a particle size no more than 100 μm. Advantageously, at least 50% by volume of the particles of the material have a particle size in the range of 5 μm to 40 μm. More advantageously, at least 50% by volume of the particles of the material have a particle size in the range of 10 to 25 μm.

Powder having a narrow range of particle size should especially be mentioned. Particle size distribution may be quoted, for example, in terms of the Geometric Standard Deviation (“GSD”) ratios d₉₀/d₅₀ or d₅₀/d₁₀ where d₉₀ denotes the particle size at which 90% by volume of the particles are below this figure (and 10% are above), d₁₀ represents the particle size at which 10% by volume of the particles are below this figure (and 90% are above) , and d₅₀ represents the mean particle size. Advantageously, the mean (d₅₀) is in the range of from 5 to 40 μm, for example, from 10 to 25 μm. Preferably, d₉₀/d₅₀ is no more than 1.5, especially no more than 1.35, more especially no more than 1.32, for example in the range of from 1.2 to 1.5, especially 1.25 to 1.35, more especially 1.27 to 1.32, the particle sizes being measured, for example, by Coulter Counter. Thus, for example, the powder may have d₅₀=10 μm, d₉₀=13 μm, d₁₀=7 μm, so that d₉₀/d₅₀=1.3 and d₅₀/d₁₀=1.4.

The powder material is fusible so that it is treatable to form a continuous film coating.

It is important that the powder can be fused or treated without degradation of any active material in the powder and without degradation of the tablet core. For some materials it may be possible for the treatment step to involve temperatures up to and above 250° C. Preferably, however, the powder material is fusible at a pressure of less than 100 lb/sq. inch, preferably at atmospheric pressure, at a temperature of less than 200° C., and most commonly below 150° C., and often at least 80° C., for example in the range of from 100 to 140° C.

Fusing of the powder material may be carried out by any of a number of different fusing methods. If desired, rupture of the shell and fusing of the material may be carried out in a single step. The powder material is preferably fused by changing the temperature of the powder, for example by radiant fusing using electromagnetic radiation, for example infra red radiation or ultra-violet radiation, or conduction or induction, or by flash fusing. The amount of heat required may be reduced by applying pressure to the powder material, for example by cold pressure fusing or hot roll fusing.

Preferably, the powder material has a glass transition temperature (Tg) in the range of 40° C. to 120° C. Advantageously, the material has a Tg in the range of 50° C. to 100° C. A preferred minimum Tg is 55° C., and a preferred maximum Tg is 70° C. Accordingly, more advantageously, the material has a Tg in the range of 55° C. to 70° C. Generally, the powder material should be heated to a temperature above its softening point, and then allowed to cool to a temperature below its Tg.

The powder material once fused is substantially insoluble, preferably completely insoluble in aqueous media at temperatures up to the body temperature. Thus, the powder material will comprise a significant amount of an insoluble material. Preferred material comprises a polymer resin selected from polymethacrylates, polyvinyl alcohols and esters, cellulose and its derivatives, cellulose ethers and esters and cellulose acetate phthalate.

The electrostatic coating of the tablet core by the powder material may be conducted by any of the methods disclosed in the above referenced patents. The partial coating of the tablet core may be achieved by the use of a mask. However, preferably the partial coating is achieved by coating one face and the sides of a tablet core in accordance with the first stage of coating as described in the above mentioned patents. Thereafter the electrostatically deposited powder is fused to form a tablet core having a casing covering one face and the sides, leaving the other face exposed.

The invention will be illustrated by the following examples and drawings in which:

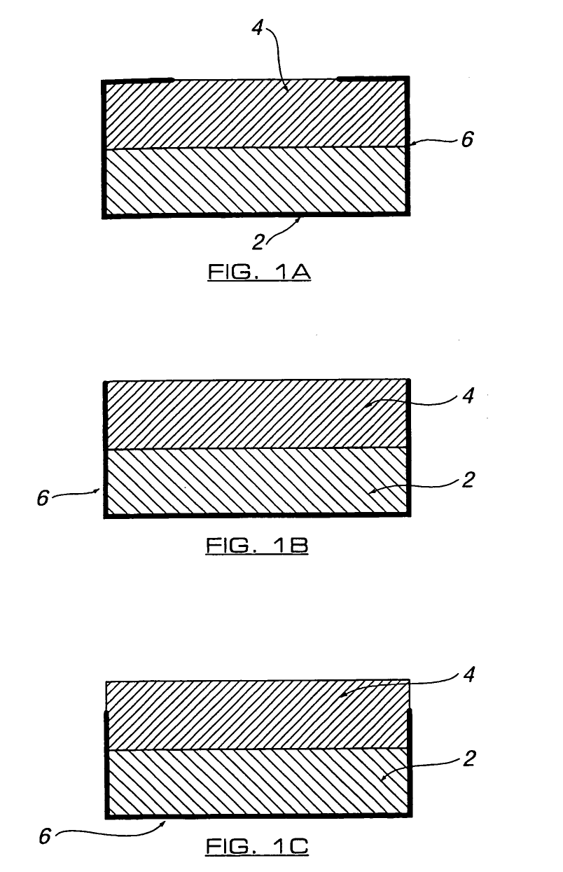

FIGS. 1a-1c show the construction of the dosage forms according to this invention.

FIGS. 2-4 show the release profile of a coated bilayer tablet providing increased rate of release

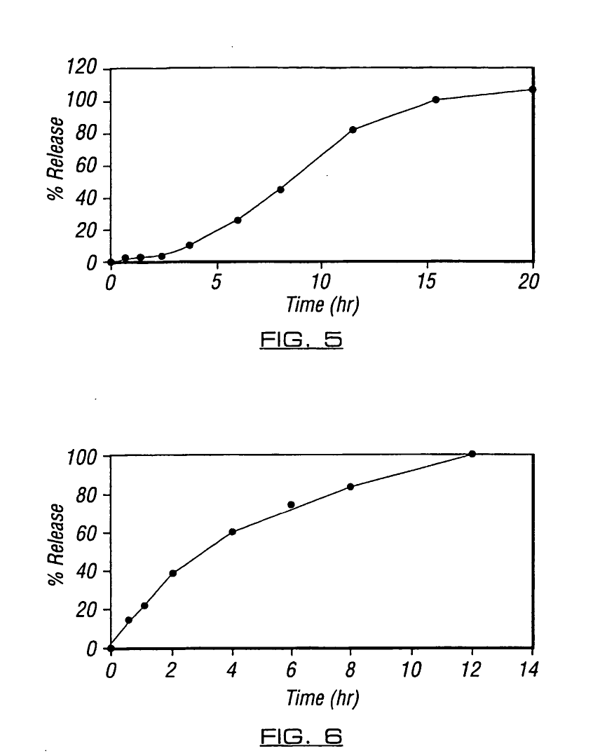

FIG. 5 shows the release profile of a coated bilayer tablet providing delayed release of salbutamol

FIG. 6 shows the release profile of a coated bilayer tablet providing an initial burst followed by sustained release of salbutamol

FIGS. 1a to c represent cross-sections through controlled release dosage forms in accordance with the invention. The dosage forms comprise an enclosed layer (2), and exposed layer (4) and an insoluble casing (6). In FIG. 1a one major surface of the exposed layer (4) is in contact with the enclosed layer (2) and the sides and a portion of the other major surface covered by the casing (6) leaving a portion of the major surface exposed. In FIG. 1b the entire surface area of a major surface of the exposed layer (4) is free of casing (6) and exposed. In FIG. 1c the entire major surface of the exposed layer and an area of the side is free of casing (6) and exposed. In all these embodiments the enclosed layer (2) is surrounded by the casing (6) and exposed layer (4).

The following materials were used in the Examples:

| | |
|---|---|
| Methocel K4M | hydroxy propyl methyl cellulose commercially available |
| | from Dow Chemicals |
| Methocel | hydroxy propyl methyl cellulose commercially available |
| K15M | from Dow Chemicals |
| Eudragit RSPO | a methacrylate copolymer commercially available from |
| | Rohm |
| Kollidone S630 | povidone from International Speciality Products |

### EXAMPLE 1

### Bilayer Tablet Having Increased Release Rate of Salbutamol Sulphate

The construction of the dosage form is shown in FIG. 1b.

Two layer tablet cores were formulated as follows:

Exposed layer formulation:

| | |
|---|---|
| Salbutamol sulphate | 0.69% |
| Methocel K4M | 15.00% |
| Anhydrous lactose DC | 83.30% |
| Magnesium stearate | 1.00% |

Enclosed layer formulation:

| | |
|---|---|
| Salbutamol sulphate | 4.82% |
| Eudragit RSPO | 10.00% |
| Anhydrous lactose DC | 84.15% |
| Magnesium stearate | 1.00% |

Approximately 175 mg of the enclosed layer formulation was added to a 10 mm die of a Manesty F3 tablet press and slightly compacted with a 10 mm normal concave punch. 175 mg of the exposed layer formulation was added to the die and the two layers compressed to form 10 mm normal biconvex tablets of hardness approximately 20 kp.

The coat formulation for the casing was as follows:
84.0% Eudragit RSPO
8.5% polyethylene glycol 6000
5.0% titanium dioxide
2.5% sunset yellow lake.

To prepare the coating powder, the above ingredients were weighed, blended, and then extruded. The extrudates were pin-milled, micronised and classified in an air jet mill to give a median particle size of approximately 10 μm.

A blend containing 4.5% of coat formulation and 95.5% of a silicone-coated ferrite was prepared. The tablets were coated electrostatically using the coat/carrier blend in a conventional dual component delivery device adapted from the electrophotographic industry such that the coating formulation (without ferrite carrier) was applied to one face and the sides of the tablet leaving the face of the exposed layer uncoated. Details of the coating process are disclosed in British Patent Application No. 9929946.3. The coat was fused onto the tablets at approximately 100° C., to provide a range of coating thickness between 20 and 50 microns.

Six tablets were assessed for release rate in 500 ml of demineralised water at 37° C. using USP apparatus II (paddles) at 50 rpm and the dissolved salbutamol was analysed using reverse phase HPLC with a UV detector at 276 nm. The release rate with time is shown in FIG. 2, which has evidently demonstrated the increasing release rate profile.

It is of interest to note that the release of salbutamol largely follows biphasic behaviour, i.e. an initial slow rate at approximately 3.6% per hour, followed by a rapid release phase at 10.0% per hour representing an in crease of 178% in release rate. The initial slow release phase extends to about 2 hours.

### EXAMPLE 2

### Bilayer Tablet Having Increased Release Rate of Salbutamol Sulphate

The construction of the dosage form is as illustrated in FIG. 1b.

Two layer tablet cores were formulated as follows:

Exposed layer formulation:

| | |
|---|---|
| Salbutamol sulphate | 1.38% |
| Methocel K15M | 15.00% |
| Anhydrous lactose DC | 82.65% |
| Magnesium stearate | 1.00% |

Enclosed layer formulation:

| | |
|---|---|
| Salbutamol sulphate | 4.13% |
| Methocel K15M | 10.00% |
| Anhydrous lactose DC | 84.85% |
| Magnesium stearate | 1.00% |

Approximately 175 mg of the enclosed layer formulation was added to a 10 mm die of a Manesty F3 tablet press and slightly compacted with a 10 mm normal concave punch. 175 mg of the exposed layer formulation was added to the die and the two layers compressed to form 10 mm normal biconvex tablets of hardness approximately 20 kp.

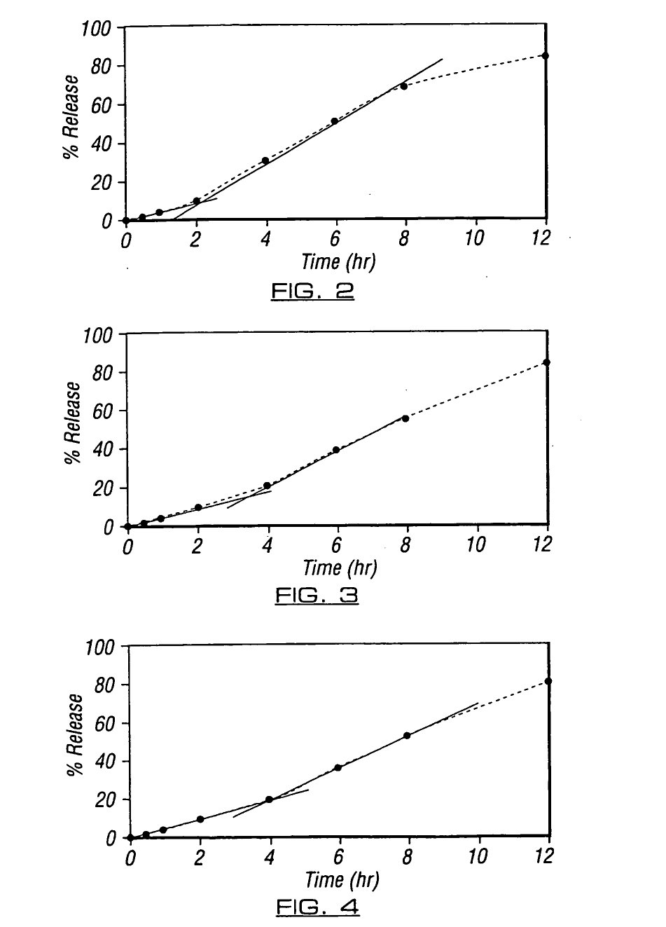

The tablet cores were coated using the materials and method described in Example 1. The release rate with time was determined for the coated tablets using the method described in Example 1 and is shown in FIG. 3.

It is evident that the electrostatic coated bilayer tablet exhibits an increased rate of release during dissolution. The release rate at the initial phase was approximately 4.5% per hour and 9.0% per hour at the later phase representing an increase of 100% in release rate. The initial release phase extends to about 3.5 hours.

### EXAMPLE 3

### Bilayer Tablet Having Increased Release Rate of Salbutamol Sulphate

The construction of the dosage form is as shown in FIG. 1b.

Two layer tablet cores were formulated as follows:

Exposed layer formulation:

| | |
|---|---|
| Salbutamol sulphate | 0.54% |
| Kolloidone S630 | 30.00% |

Dihydrogen calcium phosphate anhydrous

| | |
|---|---|
| (DCPA) | 61.86% |
| Potassium chloride | 5.00% |
| Magnesium stearate | 2.00% |
| Silicon dioxide | 0.50% |
| Indigo dye | 0.10% |

Enclosed layer formulation:

| | |
|---|---|
| Salbutamol sulphate | 3.75% |
| Kolloidone S630 | 10.00% |
| DCPA | 78.75% |
| Potassium chloride | 5.00% |
| Magnesium stearate | 2.00% |
| Silicon dioxide | 0.50% |

Two separate granules for the exposed layer formulation and the enclosed layer formulation were prepared separately. Salbutamol sulphate, potassium chloride and DCPA were sieved through 710 μm sieve, which were then blended with Salbutamol sulphate and povidone S630. The blend was then granulated with water using a Kenwood Magimix Food Processor. The wet granules were dried in a forced air oven at 60° C. to a dry matter content of less than 2.0%. The granules were screened through a 710 μm sieve and blended with dye and magnesium stearate.

Bilayer tablet cores were made by a Riva bi-layer press using 10 mm normal concave tooling. These tablet cores were coated using the materials and method described in Example 1. The release rate with time was determined for the coated tablets using the method described in Example 1 and is shown in FIG. 4.

It is evident that the electrostatic coated bilayer tablet exhibits an increased rate of release during dissolution. The release rate at the initial phase was approximately 4.6% per hour and 8.8% per hour at the later phase representing an increase of 91% in release rate. The initial release phase extends to about 4 hours.

### EXAMPLE 4

### Bilayer Tablet Having Delayed Release of Salbutamol Sulphate

The construction of the dosage form is as shown in FIG. 1b.

Two layer tablet cores were formulated as follows:

Exposed layer formulation:

| | |
|---|---|
| Salbutamol sulphate | 0.00% |
| Kolloidone S630 | 20.00% |
| DCPA | 72.40% |
| Potassium chloride | 5.00% |
| Magnesium stearate | 2.00% |
| Silicon dioxide | 0.50% |
| Indigo dye | 0.10% |

Enclosed layer formulation:

| | |
|---|---|
| Salbutamol sulphate | 4.28% |
| Kolloidone S630 | 20.00% |
| DCPA | 68.22% |
| Potassium chloride | 5.00% |
| Magnesium stearate | 2.00% |
| Silicon dioxide | 0.50% |

Two separate granules for the exposed layer formulation and the enclosed layer formulation were prepared by the same method as described in Example 3.

Bilayer tablet cores were made by a Riva bi-layer press using 10 mm normal concave tooling. These tablet cores were coated using the materials and method described in Example 1. The release rate with time was determined for the coated tablets using the method described in Example 1 and is shown in FIG. 5.

It is evident that the electrostatic coated bilayer tablet exhibited a delayed release of salbutamol with a lag time of approximately 3 hours. The release kinetics after 3 hours can be described by the following equation (up to 82% release):
% Release=10.0*(t−2.75)^{0.95}
Where t represents the dissolution time

Therefore, the subsequent release of salbutamol followed an approximately zero order release profile (when the release exponent=1.0).

### EXAMPLE 5

### Bilayer Tablet Having an Initial Burst Followed by a Constant Release Profile

The construction of the dosage form is as shown in FIG. 1b.

Two layer tablet cores were formulated as follows:

Exposed layer formulation:

| | |
|---|---|
| Salbutamol sulphate | 2.14% |
| DCPA | 42.36% |
| Microcrystalline cellulose | 10.00% |
| Lactose DC | 37.00 |
| PVP C15 | 2.00% |
| Potassium chloride | 5.00% |
| Magnesium stearate | 1.00% |
| Silicon dioxide | 0.50% |

Enclosed layer formulation:

| | |
|---|---|
| Salbutamol sulphate | 2.14% |
| Kolloidone S630 | 10.00% |
| DCPA | 60.26% |
| Potassium chloride | 5.00% |
| Magnesium stearate | 2.00% |
| Silicon dioxide | 0.50% |
| Indigo carmine lake | 0.10% |

Two separate granules for the exposed layer formulation and the enclosed layer formulation were prepared separately by the same method as described in Example 3

Bilayer tablet cores were made by a Riva bi-layer press using 10 mm normal concave tooling. These tablet cores were coated using the materials and method described in Example 1. The release rate with time was determined for the coated tablets using the method described in Example 1 and is shown in FIG. 5.

It is evident that the release profile of the bilayer tablet exhibited an initial burst followed by sustained release. The release kinetics can be described by the following equations:

| | |
|---|---|
| % Release = 26.7 t^{0.58} | (within the 0-50% release range) |
| % Release = 50.5 + 8.75 (t − 3)^{0.85} | (within the 50-85% release range) |

It is evident that the initial release follows a first order release rate (when the exponent is approximately 0.5) and the second phase of release was approximately zero order (i.e. the exponent approaching 1). The initial release phase extends to 25% of entire release period (where 100% release was achieved).

1. A controlled release dosage form with variable release rates comprising:
1) a bilayer or multilayer tablet core in which at least one of the layers contains one or more pharmaceutically active ingredients and at least one of the layers contains one or more rate controlling polymers
2) a substantially insoluble casing extended over the tablet core covering the majority of tablet surface but leaving a portion of one layer of the tablet core exposed, forming an exposed layer, the casing resulting from electrostatic deposition of a powder comprising fusible particles onto the tablet core and fusing the particles to form a thin film.

2. A controlled release dosage form as claimed in claim 1 having increased release rates over a definite period of time, in which the exposed layer contains a lower amount of active material and/or has a slower release rate than the other layer.

3. A controlled release dosage form as claimed in claim 2 which releases the active ingredient over a first period at a slower rate than a subsequent second period.

4. A controlled release dosage form as claimed in claim 3 in which the release rate during the second period is at least 50% greater than the first period.

5. A controlled release dosage form as claimed in claim 4 in which the release rate during the second period is at least 75% greater than the first period.

6. A controlled release dosage form as claimed in claim 3 in which the first period extends to at least 2 hours.

7. A controlled release dosage form as claimed in claim 1 having a delayed release profile over a definite period of time, where the exposed layer contains no active material and contains one or more rate controlling polymers.

8. A controlled release dosage form as claimed in claim 7 in which less than 10% of the active ingredient will be released in a first period of at least 1 hour.

9. A controlled release dosage form as claimed in claim 1 which initially releases a first pharmaceutically active agent at a rapid release in a fast phase followed by the release of the same or second pharmaceutically active agent or at a slower rate in which the exposed layer is free of rate controlling polymer and contains one or more active ingredients, which can be the same or different from active ingredient(s) present in the enclosed layer and one or more rate controlling polymers are present in the enclosed layer.

10. A controlled release dosage form as claimed in claim 9 in which the fast phase is completed within 40% of the entire dissolution period of the dosage form.

11. A controlled release dosage form as claimed in claim 1 in which at least 70% of at least one active ingredient is achieved after a period of 6 hours.

12. A controlled release pharmaceutical dosage form as claimed in claim 1 in which the insoluble casing covers from 65 to 95% of the surface area of the tablet core.

13. A controlled release pharmaceutical dosage form as claimed in claim 1 in which the tablet core is formed of two layers and comprises two major opposing surfaces separated by one or more sidewalls at least one major surface and the one or more sidewalls being covered by the casing.

14. A controlled release pharmaceutical dosage form as claimed in claim 1 in which at least one layer of the tablet core comprises a binder selected from acacia, alginic acid, carboxymethylcellulose, hydroxyethylcellulose, hydroxypropylcellulose, dextrin, ethylcellulose, gelatin, glucose, guar gum, hydrogenated vegetable oil, hydroxypropylmethylcellulose magnesium aluminium silicate, Maltodextrin, methylcellulose, polyethylene oxide, povidone, sodium alginate and hydrogenated vegetable oils.

15. A controlled release pharmaceutical dosage form as claimed in claim 1 in which at least one layer of tablet core additionally comprises a release rate controlling polymer selected from polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, and zein.

16. A controlled release pharmaceutical dosage as claimed in claim 1 in which at least one layer of the tablet core additionally comprises a diluent selected from lactose, cellulose, dicalcium phosphate, sucrose, dextrose, fructose, xylitol, mannitol, sorbitol, calcium sulphate, starches, calcium carbonate, sodium carbonate, dextrates, dextrin, kaolin, lactitol, magnesium carbonate, magnesium oxide, maltitol, maltodextrin and maltose.

17. A controlled release pharmaceutical dosage as claimed in claim 1 in which at least one layer of the tablet core comprises a hydrophobic matrix, a hydrophilic matrix, or a mixture of hydrophilic and hydrophobic materials.

18. A controlled release pharmaceutical dosage as claimed in claim 1 in which the active ingredient is selected from acid-peptic and motility influencing agents, laxatives, antidiarrheials, colorectal agents, pancreatic enzymes and bile acids, antiarrhythmics, antianginals, diuretics, anti-hypertensives, anti-coagulants, anti-thrombotics, fibrinolytics, haemostatics, hypolipidaemic agents, anti-anaemia and neurotropenia agents, hypnotics, anxiolytics, anti-psychotics, anti-depressants, anti-emetics, anti-convulsants, CNS stimulants, analgesics, anti-pyretics, anti-migraine agents, non-steroidal anti-inflammatory agents, anti-gout agents, muscle relaxants, neuro-muscular agents, steroids, hypoglycaemic agents, hyperglycaemix agents, diagnostic agents, antibiotics, anti-fungals, anti-malarials, anti-virals, immunosuppressants, nutritional agents, vitamins, electrolytes, anorectic agents, appetite suppressants, bronchodilators, expectorants, anti-tussives, mucolytes, decongestants, anti-glaucoma agents, oral contraceptive agents, diagnostic and neoplastic agents.

19. A controlled release pharmaceutical dosage as claimed in claim 1 in which the casing comprises a polymer resin selected from polymethacrylates, cellulose and its derivatives, cellulose ethers and esters and cellulose acetate phthalate.

20. A controlled release pharmaceutical dosage as claimed in claim 1 in which the casing additionally comprises one or more adjuvants selected from opacifiers, colourants, plasticisers, flow aids and charge control materials.

21. A controlled release pharmaceutical dosage as claimed in claim 20 in which the casing comprises a plasticiser selected from polyethylene glycols, triethyl citrate, acetyltributyl citrate, acetyltriethyl citrate, tributyl citrate, diethyl phthalate, dibutyl phthalate, dimethyl phthalate, dibutyl sebacate and glyceryl monostearate.

22. A controlled release pharmaceutical dosage as claimed in claim 1 in which the casing has an average thickness of from 20 to 50 μM.

23. A controlled release pharmaceutical dosage form as claimed in claim 1 in which the casing results in a weight gain of less than 5% by weight of the tablet core.
